(19) United States
(12) Patent Application Publication
(10) Pub. No.: US 2007/0237734 A1
(43) Pub. Date: Oct. 11, 2007

(54) ANTI-WRINKLE COSMETIC COMPOSITION

(75) Inventors: Maria Dalko, Gif S/Yvette (FR); Dominique Fagot, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/733,797

(22) Filed: Apr. 11, 2007

Related U.S. Application Data
(60) Provisional application No. 60/792,328, filed on Apr. 17, 2006.

(30) Foreign Application Priority Data
Apr. 11, 2006 (FR) 06 51327

(51) Int. Cl. A61K 8/73

(52) U.S. Cl. 424/70.13; 424/401

(57) ABSTRACT

The present invention relates to adenosine derivatives and methods of treating skin using such derivatives.

### REFERENCE TO PRIOR APPLICATIONS

This application claims priority to U.S. provisional application 60/792,328, filed Apr. 17, 2006, and to French patent application 0651327, filed Apr. 11, 2006, both of which are hereby incorporated by reference in their entirety.

### FIELD OF THE INVENTION

The present invention relates to a process for the cosmetic treatment of skin, particularly wrinkled skin, comprising the topical application to the skin, of a composition comprising an adenosine derivative. It also relates to novel adenosine derivatives.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Women, and even men, currently have a tendency to wish to appear young for as long as possible and consequently wish to soften the signs of ageing of the skin, which are reflected in particular by wrinkles and fine lines. On that subject, advertising and fashion give instances of products intended to retain radiant and wrinkle-free skin for as long as possible, these being signs of young skin, all the more so as the physical appearance affects the mind and/or the morale.

To date, wrinkles and fine lines have been treated using cosmetic products comprising active principles which act on the skin, for example by improving its cell replacement or alternatively by promoting the synthesis, or by preventing the decomposition, of the elastic fibres of which skin tissue is composed.

Although these treatments make it possible to act on wrinkles and fine lines due to chronological or intrinsic ageing and on those due to photoageing, they do not have an effect on expression wrinkles, which require operating on the muscular contractile component (via muscle-relaxing agents) or dermal contractile component (via dermo-decontracting agents) of the wrinkles.

This is because expression wrinkles are the result of different mechanisms from those which generate wrinkles due to ageing.

Specifically, they are produced under the effect of the stress exerted on the skin by the platysmas which make facial expressions possible. Depending on the shape of the face, the frequency of facial expressions and possible tics, they can appear from infancy. Age, as well as certain environmental factors, such as exposure to the sun, are not involved in their genesis but may deepen them further and render them permanent.

Expression wrinkles are characterized by the presence of grooves around the orifices formed by the nose (nasogenian grooves), the mouth (parabuccal wrinkles and “sour-face” wrinkles) and the eyes (crows feet), around which lie the platysmas, and between the eyebrows (glabella wrinkles or “lions” wrinkles) and on the forehead.

To date, the only means commonly used for acting on the expression wrinkles is botulinum toxin, which is injected in particular into the glabella wrinkles, which are wrinkles between the eyebrows (see J. D. Carruters et al., J. Dermatol. Surg. Oncol., 1992, 18, pp. 17-21).

In addition, the Applicant Company has provided various compounds capable of offering an anti-wrinkle effect when they are applied topically to the skin, thus making it possible to act via another route on expression wrinkles.

However, the need remains to have available other compounds effective in smoothing out or softening wrinkles, in particular expression wrinkles.

In point of fact, the Applicant Company has discovered, with astonishment, that certain adenosine derivatives make it possible to satisfy this need.

### DETAILED DESCRIPTION OF THE PREFERRED EMBODIMENTS

As used herein, the phrases “selected from the group consisting of,” “chosen from,” and the like include mixtures of the specified materials. Terms such as “contain(s)” and the like as used herein are open terms meaning ‘including at least’ unless otherwise specifically noted.

A subject-matter of the present invention is thus the cosmetic use of at least one adenosine derivative of formula (I): 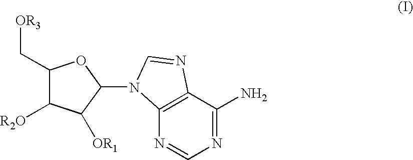
in which:

(a) R₁ and R₂, which are identical, denote a saturated linear C₁-C₆ or unsaturated linear C₂-C₆ or saturated or unsaturated branched C₃-C₆ hydrocarbon radical or form, together with the oxygen atom to which they are attached, an isopropylidene radical;

and R₃ denotes:

(i) a —COR₄ group with R₄ denoting a saturated linear C₁-C₉ or unsaturated linear C₂-C₉ or saturated or unsaturated branched C₃-C₉ hydrocarbon radical optionally substituted by at least one group chosen from —OR′, —NR′R″, —COOR′, —CONR′R″, —CF₃, —F, —OCF₃, —CN or —NO₂;

or (ii) an ester group resulting from biotin;

or
(b) R₁ and R₂ form, together with the oxygen atoms to which they are attached, an isopropylidene radical; and R₃ denotes a saturated linear C₁-C₁₀ or unsaturated linear C₂-C₁₀ or saturated or unsaturated branched C₃-C₁₀ hydrocarbon radical optionally substituted by at least one group chosen from —OR′, —NR′R″, —COOR′, —CONR′R″, —CF₃, —F, —OCF₃, —CN or —NO₂;

R′ and R″ denoting a hydrogen atom or a saturated linear C₁-C₆ or unsaturated linear C₂-C₆ or saturated or unsaturated branched C₃-C₆ hydrocarbon radical optionally substituted by at least one group chosen from —OZ, —NZZ′ or —COOZ, Z and Z′ denoting, independently of one another, a hydrogen atom or a saturated linear C₁-C₆ or unsaturated linear C₂-C₆ or saturated or unsaturated branched C₃-C₆ hydrocarbon radical;

and its salts, optical isomers and solvates,

as an agent for combating wrinkles, in particular expression wrinkles, and/or relaxing the skin and/or slackening the lines of the skin.

A further subject-matter of the invention is a process for the cosmetic treatment of skin, in particular wrinkled skin, preferably the skin of the face and/or forehead, comprising the topical application to the said skin of a composition comprising, in a physiologically acceptable medium, at least one adenosine derivative of formula (I) as defined above.

Some of the compounds of formula (I) are known from the prior art and are described in the following documents:
WO-A-2004/037159;
Poppe, L. et al.; “Synthesis and characterization of (5′-deoxyadenosin-5′-yl)cobalamin (=‘adenosyl-cobalamin’) analogs mimicking the transition-state geometry of coenzyme-B12-dependent rearrangements”; Helvetica Chimica Acta (1993), 76(6), 2367-83;
Jones, A. S. et al.; “Synthetic analogs of polynucleotides. VII. Syntheses of 5′-O-acryloylnucleosides and copolymers of these with other acryloyl compounds”; Journal of the Chemical Society [Section] C: Organic (1971), (19), 3183-7;
Mornet, D. et al.; “The reaction of myosin with a bromoalkyl analog of adenosine triphosphate”; FEBS Letters (1977), 84(2), 362-6;
Huber Gerhard; “Esters of adenosine with organic and inorganic acids”; Chem. Ber., 89, 2853-62 (1956)—ref. CA52:2027g;
Takemoto, K. et al.; “Nucleic acid analogs: their specific interaction and applicability”; Polymeric Materials Science and Engineering (1988), 58, 250-3;
Purkayastha, Bhupesh C.; Bhattacharyya, S. N.; “Use of Ca oxalate monohydrate in the investigation of rare earth and thorium activities”; J. Indian. Chem. Soc., 34, 427-33 (1957)—ref. CA52:2627h;
Peterli, Stefan et al.; “Nitrostyrene derivatives of adenosine 5′-glutarates as selective inhibitors of the epidermal growth factor receptor protein tyrosine kinase”; Helvetica Chimica Acta (1992), 75(3), 696-706.

For example, 2′,3′-Isopropylidene-5′-acetyladenosine is a known compound, described in particular in Application WO-A-2004/037159 (compound 265, page 203) in a pharmaceutical composition for the treatment of obesity. This document does not describe applying the composition topically to the skin, in particular for treating wrinkles.

Another subject-matter of the invention is a composition comprising, in a physiologically acceptable medium, at least one adenosine derivative of formula 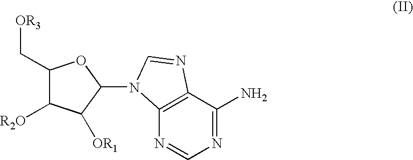
in which:
(a) R₁ and R₂, which are identical, denote a saturated linear C₁-C₆ or unsaturated linear C₂-C₆ or saturated or unsaturated branched C₃-C₆ hydrocarbon radical or form, together with the oxygen atoms to which they are attached, an isopropylidene radical;

and R₃ denotes:

(i) a —COR₄ group with R₄ denoting a saturated linear C₂-C₉ or unsaturated linear C₂-C₉ or saturated or unsaturated branched C₃-C₉ hydrocarbon radical optionally substituted by at least one group chosen from —OR′, —NR′R″, —COOR′, —CONR′RR″, —CF₃, —F, —OCF₃, —CN or —NO₂;

or (ii) an ester group resulting from biotin;

or
(b) R₁ and R₂ form, together with the oxygen atoms to which they are attached, an isopropylidene radical; and R₃ denotes a saturated linear C₁-C₁₀ or unsaturated linear C₂-C₁₀ or saturated or unsaturated branched C₃-C₁₀ hydrocarbon radical optionally substituted by at least one group chosen from —OR′, —NR′R″, —COOR′, —CONR′R″, —CF₃, —F, —OCF₃, —CN or —NO₂;

R′ and R″ denoting a hydrogen atom or a saturated linear C₁-C₆ or unsaturated linear C₂-C₆ or saturated or unsaturated branched C₃-C₆ hydrocarbon radical optionally substituted by at least one group chosen from —OZ, —NZZ′ or —COOZ, Z and Z′ denoting, independently of one another, a hydrogen atom or a saturated linear C₁-C₆ or unsaturated linear C₂-C₆ or saturated or unsaturated branched C₃-C₆ hydrocarbon radical;

and its salts, optical isomers and solvates.

A further subject-matter of the invention is novel compounds of formula (III): 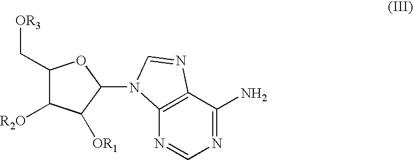
in which:
(a) R₁ and R₂, which are identical, denote a saturated linear C₁-C₆ or unsaturated linear C₂-C₆ or saturated or unsaturated branched C₃-C₆ hydrocarbon radical or else form, together with the oxygen atoms to which they are attached, an isopropylidene radical;
R₃ denotes:

(i) a —COR₄ group with R₄ denoting a linear C₃-C₉ hydrocarbon radical or a branched C₃-C₉ hydrocarbon radical which is saturated or unsaturated, optionally substituted by at least one group chosen from —OR′, —NR′R″, —CONR′R″, —CF₃, —F, —OCF₃, —CN or —NO₂, R₄ not denoting a 2-aminoethyl group;

or (ii) an ester group resulting from biotin;

or
(b) R₁ and R₂ form, together with the oxygen atoms to which they are attached, an isopropylidene radical; and R₃ denotes a linear C₂-C₁₀ hydrocarbon radical or a branched C₃-C₁₀ hydrocarbon radical which is saturated or unsaturated, optionally substituted by at least one group chosen from —NR′R″, —COOR′, —CONR′R″, —CF₃, —F, —OCF₃, —CN or —NO₂;

R′ and R″ denoting a hydrogen atom, a saturated linear C₁-C₆ or unsaturated linear C₂-C₆ hydrocarbon radical or a saturated or unsaturated branched C₃-C₆ radical optionally substituted by at least one group chosen from —OZ, —NZZ′ or —COOZ, Z and Z′ denoting, independently of one another, a hydrogen atom or a saturated linear C₁-C₆ or unsaturated linear C₂-C₆ or saturated or unsaturated branched C₃-C₆ hydrocarbon radical;

and their salts, optical isomers and solvates.

In formula (I), the hydrocarbon (or alkyl) groups can preferably be chosen in particular, as the case may be, from the groups: methyl, ethyl, n-propyl, isopropyl, n-butyl, isobutyl, tert-butyl, pentyl, hexyl, heptyl, octyl, non-yl or decyl.

Radicals R₁ and R₂ which are preferred in formulae (I) to (III) described above are those forming, together with the oxygen atoms to which they are attached, an isopropylidene radical.

For the compounds of formula (I), preference is given to those having the following meanings:
R₁ and R₂ form, together with the oxygen atoms to which they are attached, an isopropylidene radical;
R₃ denotes:

(i) a saturated linear C₁-C₁₀ or unsaturated linear C₂-C₁₀ or saturated or unsaturated branched C₃-C₁₀ hydrocarbon radical;

or (ii) a —COR₄ group with R₄ denoting a linear C₁-C₉ hydrocarbon radical or a branched C₃-C₉ hydrocarbon radical which is saturated or unsaturated; or (iii) an ester group resulting from biotin.

Preferably, in formula (I):
R₁ and R₂ form, together with the oxygen atoms to which they are attached, an isopropylidene radical;
R₃ denotes:

(i) a linear C₁-C₁₀ hydrocarbon radical;

or (ii) a —COR₄ group with R₄ denoting a saturated linear C₁-C₉ hydrocarbon radical;

or (iii) an ester group resulting from biotin.

More preferably, in formula (I):

R₁ and R₂ form, together with the oxygen atoms to which they are attached, an isopropylidene radical; R₃ denotes:
a —COR₄ group with R₄ denoting a linear C₁-C₉ hydrocarbon radical;
or an ester group resulting from biotin.

For the compounds of formula (II), preference is given to those having the following meanings:

R₁ and R₂ form, together with the oxygen atoms to which they are attached, an isopropylidene radical;

R₃ denotes:

(i) a saturated linear C₁-C₁₀ or unsaturated linear C₂-C₁₀ or saturated or unsaturated branched C₃-C₁₀ hydrocarbon radical;

or (ii) a —COR₄ group with R₄ denoting a linear C₂-C₉ hydrocarbon radical or a branched C₃-C₉ hydrocarbon radical which is saturated or unsaturated;

or (iii) an ester group resulting from biotin.

Preferably, in formula (II):

R₁ and R₂ form, together with the oxygen atoms to which they are attached, an isopropylidene radical;

R₃ denotes:

(i) a linear C₁-C₁₀ hydrocarbon radical;

or (ii) a —COR₄ group with R₄ denoting a saturated linear C₂-C₉ hydrocarbon radical;

or (iii) an ester group resulting from biotin.

More preferably, in formula (II):

R₁ and R₂ form, together with the oxygen atoms to which they are attached, an isopropylidene radical;

R₃ denotes:
a —COR₄ group with R₄ denoting a linear C₂-C₉ hydrocarbon radical;
or an ester group resulting from biotin.

For the compounds of formula (III), preference is given to those having the following meanings:

R₁ and R₂ form, together with the oxygen atoms to which they are attached, an isopropylidene radical;

R₃ denotes:

(i) a linear C₂-C₁₀ hydrocarbon radical or a branched C₃-C₁₀ hydrocarbon radical which is saturated or unsaturated;

or (ii) a —COR₄ group with R₄ denoting a linear C₃-C₉ hydrocarbon radical or a branched C₃-C₉ hydrocarbon radical which is saturated or unsaturated;

or (iii) an ester group resulting from biotin.

Preferably, in formula (III):

R₁ and R₂ form, together with the oxygen atoms to which they are attached, an isopropylidene radical;

R₃ denotes:

(i) a linear C₂-C₁₀ hydrocarbon radical;

or (ii) a —COR₄ group with R₄ denoting a saturated linear C₃-C₉ hydrocarbon radical;

or (iii) an ester group resulting from biotin.

More preferably, in formula (III):

R₁ and R₂ form, together with the oxygen atoms to which they are attached, an isopropylidene radical;

R₃ denotes:
a —COR₄ group with R₄ denoting a linear C₃-C₉ hydrocarbon radical;
or an ester group resulting from biotin.

Mention may be made, as salts of the compounds of formulae (I) to (III), of the salts obtained by addition of compound of formula (I), (II) or (III) with an inorganic acid, preferably chosen in particular from hydrochloric acid, sulphuric acid and phosphoric acid, or with an organic acid, chosen in particular from acetic acid, propionic acid, succinic acid, fumaric acid, lactic acid, glycolic acid, citric acid and tartaric acid.

Preferably, the salts of the compounds (I), (II) or (III) are chosen from the salts obtained from hydrochloric acid or acetic acid or citric acid.

The compounds (I), (II) or (III) can preferably be prepared according to one of the four synthetic processes described below depending on the meaning of the radicals R₁, R₂ and R₃.

First Process

The compounds of formula (I) for which R₁═R₂ and R₃ denote a hydrocarbon radical as defined above can preferably be prepared in particular according to the following reaction scheme I:

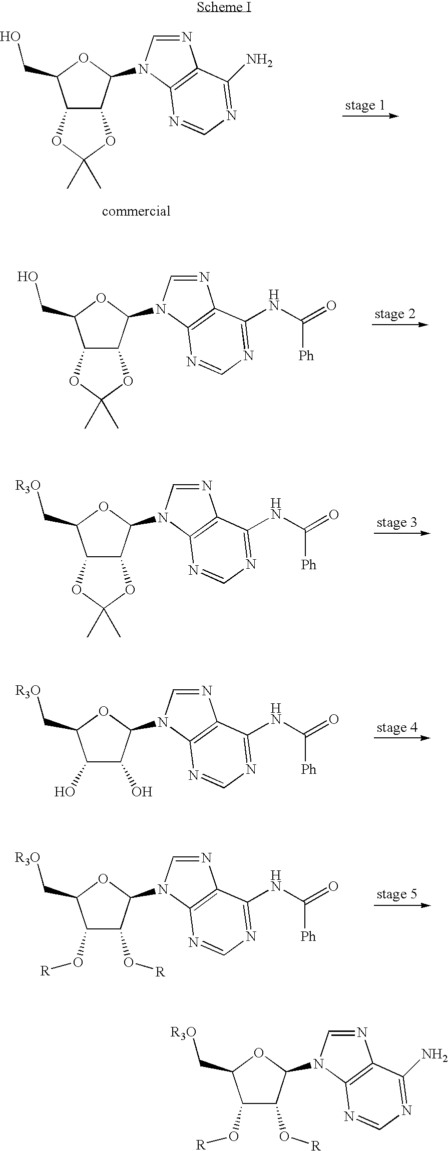

Such a preparation method is described in particular in Helvetica Chimica Acta, 1993 (vol. 76), page 2367.

According to stage 1, isopropylideneadenosine is reacted with chlorotrimethylsilane, in particular in pyridine, and then 1.2 molar equivalents of benzoyl chloride are added. After formation of the corresponding N-benzoyl derivative, sodium fluoride in a water/methanol mixture in an acidic medium is added.

According to stage 2, sodium hydride in dimethylformamide is added to the compound obtained, and a tosylate of formula R₃OTs (Ts denotes the tosyl group) or a halogen compound of formula R₃X (with X denoting Cl, Br or I) is added.

According to stage 3, a 10% aqueous hydrochloric acid solution and methanol are added and the mixture is brought to reflux for 10 minutes.

According to stage 4, sodium hydride in dimethylformamide and then 2 molar equivalents of tosylate compound of formula R₃OTs (Ts denotes the tosyl group) or of halogen compound of formula R₃X (with X denoting Cl, Br or I) are added.

According to stage 5, a catalytic amount of sodium methoxide in methanol is added.

Second Process

The compounds of formula (I) for which R₁ and R₂ together form an isopropylidene radical and R₃ denotes a hydrocarbon radical as defined above can preferably be prepared in particular according to the following reaction scheme II:

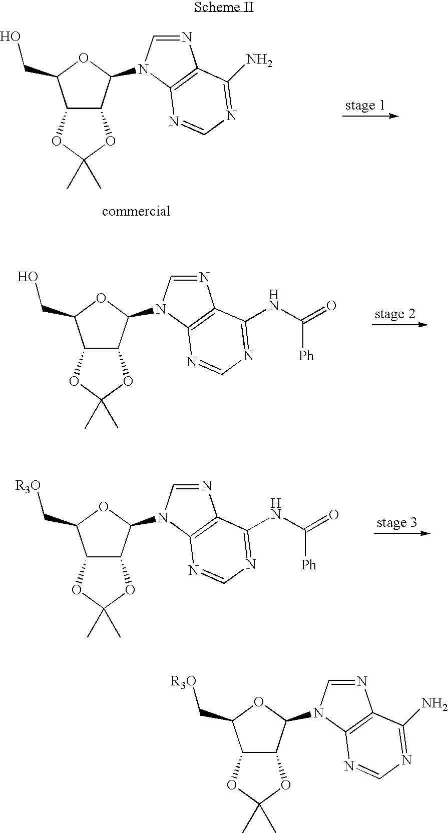

According to stage 1, isopropylideneadenosine is reacted with chlorotrimethylsilane, in particular in pyridine, and then 1.2 molar equivalents of benzoyl chloride are added. After formation of the corresponding N-benzoyl derivative, sodium fluoride in a water/methanol mixture in an acidic medium is added.

According to stage 2, sodium hydride in dimethylformamide is added to the compound obtained and a tosylate of formula R₃OTs (Ts denotes the tosyl group) or a halogen compound of formula R₃X (with X denoting Cl, Br or I) is added,

According to stage 3, a catalytic amount of sodium methoxide in methanol is added.

Third Process

The compounds of formula (I) for which R₁═R₂ and denote a hydrocarbon radical and R₃ denotes a —COR₄ radical or an ester group resulting from biotin as defined above can preferably be prepared in particular according to the following reaction scheme III:

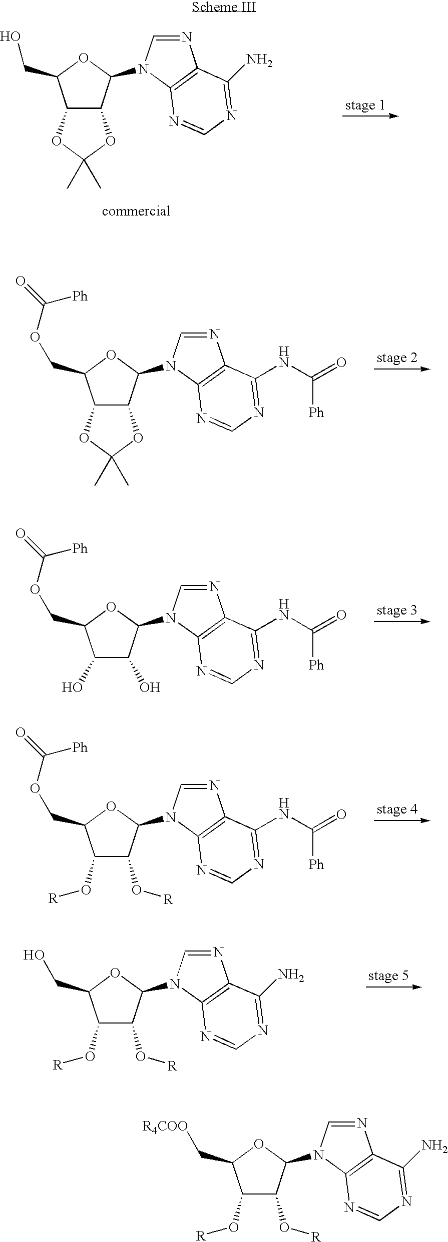

According to stage 1, isopropylideneadenosine is reacted with 2.1 molar equivalents of benzoyl chloride, in particular in pyridine.

Then, according to stage 2, a 10% aqueous hydrochloric acid solution and methanol are added and the mixture is brought to reflux for 10 minutes.

According to stage 3, sodium hydride in dimethylformamide and then 2 molar equivalents of halogen compound of formula R₃X (with X denoting Cl, Br or I) are added.

According to stage 4, a catalytic amount of sodium methoxide in methanol is added.

According to stage 5, an organic acid of formula R₄COOH is added in the presence of carbonyldiimidazole in dimethylformamide at a temperature of approximately 40° C.

Fourth Process

The compounds of formula (I) for which R₁ and R₂ together form an isopropylidene radical and R₃ denotes a —COR₄ radical or an ester group resulting from biotin as defined above can preferably be prepared in particular according to the following reaction scheme IV:

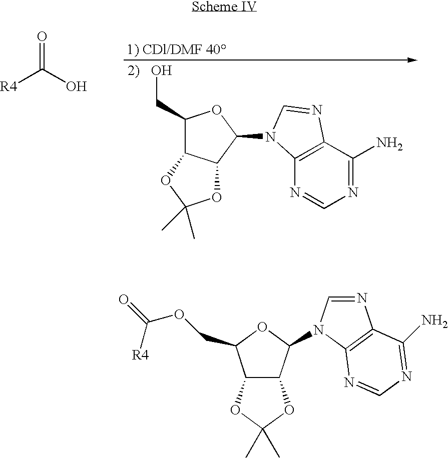

The carboxylic acid R₄COOH is reacted in the presence of carbodiimide (CDI) in dimethylformamide at a temperature of approximately 40° C. and isopropylideneadenosine is added.

Mention may be made, as novel compounds of formula (III) (which also form part of the compounds of formulae (I) and (II) described above), of the following compounds:

Compound A: 2′,3′-isopropylidene-5′-butanoyladenosine

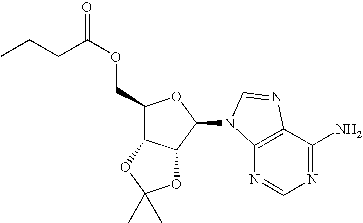

Compound B: 2′,3′-isopropylidene-5′-octanoyladenosine

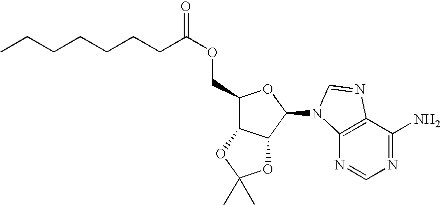

Compound C: 2′,3′-isopropylidene-5′-biotinoyladenosine

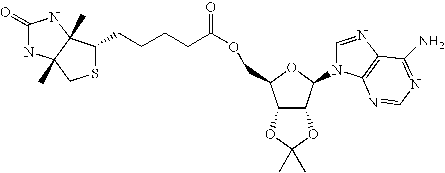

Compound D: 2′,3′-isopropylidene-5′-ethyladenosine

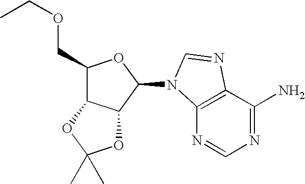

Compound E: 2′,3′-isopropylidene-5′-octyladenosine

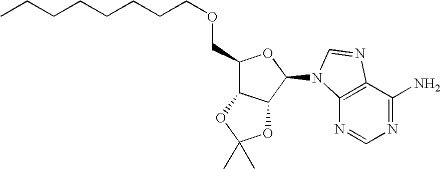

Compound F: 2′,3′-dimethyl-5′-butanoyladenosine

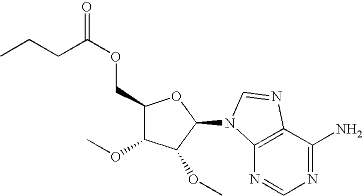

Mention may be made, as known compounds forming part of the compounds of formula (I), of:

Compound G: 2′,3′-isopropylidene-5′-acetyladenosine (CAS No. 15888-38-7)

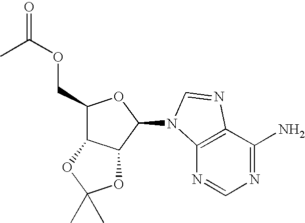

The amount of adenosine derivative which can be used according to the invention depends, of course, on the effect desired and can thus vary widely.

In this regard, the invention method and composition is preferably used by subjects desirous of the benefits noted herein, subjects “in need of” these benefits. Such subjects are typically suffering from signs of expression lines or wrinkles determined by, for example, self diagnosis or cosmetician or medical diagnosis, or are at recognized and appreciated risk of developing such conditions and who use the invention methods and compositions to combat these effects. In this regard, the invention process can be viewed as one for delaying the onset of the appearance of, and/or for reducing signs of, expression lines or wrinkles.

Naturally, one using the invention as disclosed will use an amount of the invention composition effective to reduce the signs of expression lines or wrinkles. Such amount is inclusive of an amount sufficient to effect muscle relaxation and/or dermo-contraction and is further inclusive of the compositions described herein at the disclosed concentrations of active ingredients sufficient to cover the area of the skin being treated in a single application, and of course includes that amount applied upon repeated application, for example on a daily basis over a course of days, weeks, etc. In a preferred embodiment the invention process includes multiple applications of the invention composition to the area(s) of skin in need of attention.

To give a representative order of magnitude, these derivatives can preferably be used in an amount representing from 0.01% to 10% of the total weight of the composition, more preferably in an amount representing from 0.05% to 5% of the total weight of the composition, and more preferably in an amount representing from 0.1% to 2% of the total weight of the composition.

The composition according to the invention is suitable for topical application to the skin and it thus comprises a physiologically acceptable medium, that is to say a medium compatible with the skin and optionally with its superficial body growths (eyelashes, nails, hair) and/or the mucous membranes. This medium is advantageously cosmetically acceptable, that is to say it does not result in itching, smarting or redness liable to dissuade the user from using the composition and it exhibits a pleasant appearance, smell and feel.

This composition can be provided in all the pharmaceutical forms normally used in the cosmetics field and it can in particular be in the form of an optionally gelled solution, an optionally two-phase dispersion of the lotion type, an emulsion obtained by dispersion of a fatty phase in an aqueous phase (O/W) or vice versa (W/O), or a triple (W/O/W or O/W/O) emulsion or a vesicular dispersion of ionic and/or non-ionic type. These compositions are prepared according to the usual methods. It is preferable to use, according to this invention, a composition in the form of an oil-in-water emulsion.

This composition can be more or less fluid and have the appearance of a white or coloured cream, ointment, milk, lotion, serum, paste or foam. It can optionally be applied in the aerosol form. It can also be provided in the solid form, in particular in the stick form. It can be used as care product and/or as makeup product for the skin.

In a known way, the composition used according to the invention can also comprise the adjuvants usual in the cosmetics field, such as oils, waxes, emulsifiers, gelling agents, film-forming polymers, preservatives, fragrances, fillers, UV screening agents, bactericides, odour absorbers, dyestuffs, hydrophilic or lipophilic active principles, plant extracts or antioxidants. The amounts of these various adjuvants are those conventionally used in the field under consideration, for example from 0.01 to 20% of the total weight of the composition. These adjuvants, depending on their nature, can be introduced into the fatty phase, into the aqueous phase or into the lipid vesicles. In any case, these adjuvants and their proportions will be chosen so as not to harm the properties desired for the compounds according to the invention.

When the composition used according to the invention is an emulsion, the proportion of the fatty phase can range from 5 to 80% by weight, and preferably from 5 to 50% by weight, with respect to the total weight of the composition. The oils, the emulsifiers and the coemulsifiers used in the composition in the emulsion form are chosen from those conventionally used in the field under consideration. The emulsifier and the coemulsifier are present in the composition in a proportion ranging from 0.3 to 30% by weight, and preferably from 0.5 to 20% by weight, with respect to the total weight of the composition.

Mention may be made, as oils which can be used in the invention, of mineral oils (liquid petrolatum), oils of vegetable origin (avocado oil, soybean oil), oils of animal origin (lanolin), synthetic oils (perhydro-squalene), silicone oils (cyclomethicone) and fluorinated oils (perfluoropolyethers). Use may also be made, as fatty substances, of fatty alcohols (cetyl alcohol), fatty acids or waxes (carnauba wax, ozokerite).

Mention may be made, as emulsifiers and coemulsifiers which can be used in the invention, for example, of esters of fatty acid and of polyethylene glycol, such as PEG-100 stearate, and esters of fatty acid and of glycerol, such as glyceryl stearate.

Mention may in particular be made, as hydrophilic gelling agents/thickening agents, of carboxyvinyl polymers (carbomer), acrylic copolymers, such as acrylate/alkylacrylate copolymers, polyacrylamides, polysaccharides, natural gums and clays, and mention may be made, as lipophilic gelling agents/thickening agents, of modified clays, such as bentones, metal salts of fatty acids and hydrophobic silica.

As active principles, it will be advantageous to introduce, into the composition used according to the invention, at least one compound chosen from: desquamating agents; moisturizing agents; antiglycation agents; NO-synthase inhibitors; agents which stimulate the synthesis of dermal or epidermal macromolecules and/or which prevent their decomposition; agents which stimulate the proliferation of fibroblasts and/or of keratinocytes or which stimulate the differentiation of keratinocytes; other muscle-relaxing agents and/or dermo-decontracting agents; tightening agents; agents for combating pollution or free radicals; agents which act on the microcirculation; agents which act on the energy metabolism of cells; and their mixtures.

Examples of such additional compounds are: retinol and its derivatives, such as retinyl palmitate; ascorbic acid and its derivatives, such as magnesium ascorbyl phosphate and ascorbyl glucoside; tocopherol and its derivatives, such as tocopheryl acetate; nicotinic acid and its precursors, such as nicotinamide; ubiquinon-e; glutathione and its precursors, such as L-2-oxothiazolidine-4-carboxylic acid; plant extracts and in particular plant proteins and their hydrolysates, and also phytohormones; marine extracts, such as algal extracts; bacterial extracts; sapogenins, such as diosgenin, and the wild yam extracts comprising the latter; ceramides; hydroxy acids, such as salicylic acid and 5-(n-octanoyl)salicylic acid; resveratrol; oligopeptides and pseudopeptides and their acylated derivatives; manganese and magnesium salts, in particular gluconates; and their mixtures.

As indicated above, the composition according to the invention can also include photoprotective agents active in the UW-A and/or UW-B regions, in the form of organic or inorganic compounds, the latter optionally being coated in order to render them hydrophobic.

The organic photoprotective agents can be chosen in particular from: anthranilates, in particular menthyl anthranilate; benzophenones, in particular benzo-phenone-1, benzophenone-3, benzophenone-5, benzo-phenone-6, benzophenone-8, benzophenone-9, benzo-phenone-12 and preferably benzophenone-3 (oxybenzone) or benzophenone-4 (Uvinul MS40, available from BASF); benzylidenecamphors, in particular 3-benzylidene camphor, benzylidene camphor sulphonic acid, camphor benzalkonium methosulphate, polyacrylamidomethyl benzylidene camphor, terephthalylidene dicamphor sulphonic acid and, preferably, 4-methylbenzylidene camphor (Eusolex 6300, available from Merck); benzimidazoles, in particular benzimidazilate (Neo Heliopan AP, available from Haarmann and Reimer) or phenylbenzimidazole sulphonic acid (Eusolex 232, available from Merck); benzotriazoles, in particular drometrizole trisiloxane or methylene bis-benzotriazolyl tetramethylbutylphenol (Tinosorb M, available from Ciba); cinnamates, in particular cinoxate, DEA methoxycinnamate, diisopropyl methyl cinnamate, glyceryl ethylhexanoate dimethoxycinnamate, isopropyl methoxycinnamate, isoamyl cinnamate and preferably ethocrylene (Uvinul N35, available from BASF), octyl methoxycinnamate (Parsol MCX, available from Hoffmann-LaRoche) or octocrylene (Uvinul 539, available from BASF); dibenzoylmethanes, in particular butyl methoxydibenzoylmethane (Parsol 1789); imidazolines, in particular ethylhexyl dimethoxy-benzylidene dioxoimidazoline; PABAs, in particular ethyl dihydroxypropyl PABA, ethylhexyl dimethyl PABA, glyceryl PABA, PABA, PEG-25 PABA and preferably diethylhexyl butamido triazone (Uvasorb HEB, available from 3V Sigma), ethylhexyl triazone (Uvinul T150, available from BASF) or ethyl PABA (benzocaine); salicylates, in particular dipropylene glycol salicylate, ethylhexyl salicylate, homosalate or TEA salicylate; triazines, in particular anisotriazine (Tinosorb S, available from Ciba); or drometrizole trisiloxane.

The inorganic photoprotective agents are preferably composed of zinc oxide and/or of titanium dioxide, preferably of nanometric size, optionally coated with alumina and/or with stearic acid.

The composition according to the invention is advantageously intended to be applied to the areas of the face and/or of the forehead marked by expression wrinkles and/or to the people exhibiting expression wrinkles.

The wrinkles concerned are preferably those positioned radially around the mouth and/or eyes, in particular crows feet, and/or situated on the forehead, in particular the “lion's” wrinkle, situated in the glabella, in the space between the eyebrows, and/or positioned horizontally on the forehead.

### EXAMPLES

The invention will now be illustrated by the following non-limiting examples. In these examples, the amounts are indicated as percentages by weight.

### Example 1

### Synthesis of 2′,3′-isopropylidene-5′-biotinoyladenosine (Compound C)

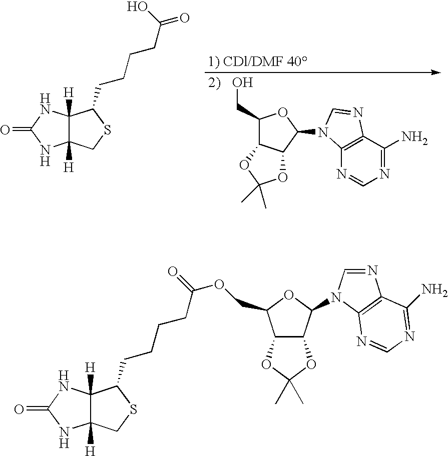

150 ml of dimethylformamide, 5 g of biotin and then 4 g of carbonyldiimidazole (denoted by CDI) were introduced into a three-necked flask under nitrogen. The mixture was heated at 40° C. for 1 hour, then 6.3 g of commercial isopropylideneadenosine were added and then 5 g of sodium amide were added. The solution was heated at 40° C. for 24 hours.

The dimethylformamide (denoted by DMF) was distilled off without exceeding 40° C. and 200 ml of dichloromethane were added to the residue.

The organic phase was washed 3 times with 150 ml of water, dried with sodium sulphate and evaporated under vacuum to dryness.

The residue was purified on a silica column, elution being carried out with dichloromethane then dichloromethane 95/methanol 5, to result in a solid product which was taken up in dichloromethane and precipitated with diethyl ether.

The precipitate obtained was filtered off, washed with ether and dried under vacuum.

Yield=50%

Analyses:

NMR DMSO ¹H ¹³C ²D: Spectra in accordance

Elemental analysis in accordance: C, 51.3; H, 5.89; N, 18.2; O, 19.03; S, 5.92

### Example 2

### Synthesis of 2′,3′-isopropylidene-5′-butanoyladenosine (Compound A)

This compound was prepared according to a procedure similar to that of Example 1 using butyric acid instead of biotin.

### Example 3

### Synthesis of 2′,3′-isopropylidene-5′-octanoyladenosine (Compound B)

This compound is prepared according to a procedure similar to that of Example 1 using octanoic acid instead of biotin.

### Example 4

### Synthesis of 2′,3′-isopropylidene-5′-ethyl-adenosine (Compound D)

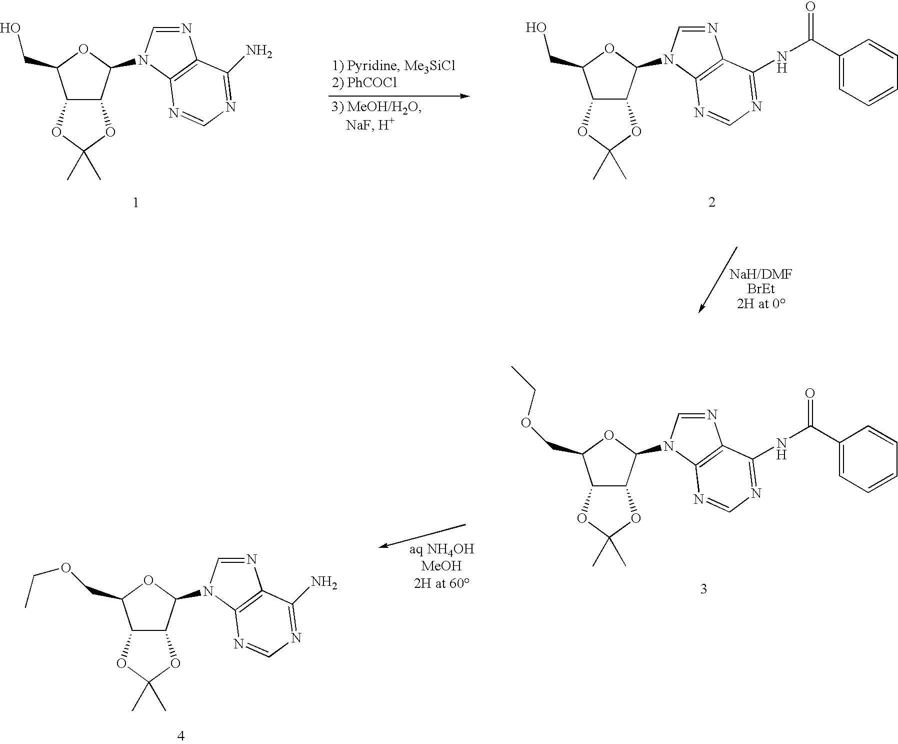 a) Chlorotrimethylsilane (1.76 g, 16.3 mmol) was added dropwise to a solution of isopropylideneadenosine (1) (4 g, 13 mmol) and 4-(dimethylamino)pyridine (50 mg) in 20 ml of pyridine at 0° C.

The mixture was stirred at 0° C. for 10 min and then at ambient temperature for 1 h.

Benzoylchloride (1.51 ml, 13 mmol) was added and the mixture was stirred at 0° C. for 10 min.

After stirring at ambient temperature for 2 hours, 30 ml of 6/4 MeOH/H₂O, 1 g of NaF and tetrabutylammonium chloride (100 mg) were added and then the mixture was left stirring at ambient temperature overnight.

The mixture was diluted with 40 ml of ice-cold water and the pH was adjusted to 2.5 with approximately 40 ml of 5N HC_{1;} after extracting with CH₂Cl₂ (4×50 ml), the organic phases were washed with 2M HCl (20 ml), then with a saturated NaHCO₃ solution (30 ml) and then with water, and dried over Na₂SO₄.

After evaporating the solvent, the residue was purified on a silica column, elution being carried out with 3/1 CH₂Cl₂/acetone.

2 g of the compound (2) were obtained in the form of a white solid.

b) A solution of the compound (2) (500 mg, 1.2 mmol) in 10 ml of DMF was cooled to 0° C., 245 mg of 60% NaH in oil (6 mmol) were added and the mixture was stirred for 30 min; 105 μl of bromoethane were added and the reaction was allowed to take place for 2 hours without removing the ice bath. The mixture was treated by addition of 15 ml of a saturated NH₄Cl solution and then extracted with CH₂Cl₂. The organic phases were washed with water and then with water saturated with NaCl, dried over Na₂SO₄ and evaporated.

650 mg of the compound (3) (corresponding to compound D) were obtained in the form of an oil which was used without further purification in the following stage.

c) 25 ml of a 20% aqueous NH₃ solution (140 mmol) were added to a solution of compound (3) (600 mg, 1.4 mmol) in 20 ml of MeOH.

The solution was heated at 60° C. for 2 hours and then, after cooling, evaporated.

The residue was purified on silica gel (3/1 CH₂Cl₂/acetone).

200 mg of the compound (4) were obtained in the form of a white solid.

Yield over the last two stages: 45%

NMR d₆-DMSO ¹H¹³C: Spectra in accordance

### Example 5

### Synthesis of 2′,3′-isopropylidene-5′-octyl-adenosine (Compound E)

This compound was prepared according to a procedure similar to that of Example 4 using octyl bromide instead of ethyl bromide.

### Example 6

Demonstration of the dermo-decontracting effect of the compounds used according to the invention.

a) Principle of the Test

The principle of this test consists in studying the effect of the test product on a dermal equivalent model composed of a collagen matrix inoculated with normal human fibroblasts.

These conditions are intended to mimic in vitro the dermal contractile phenomena which occur during facial expressions. This is because, under these conditions, the cells spontaneously express tensile forces which induce retraction of the collagen gel. This results in a decrease in the total surface area of the dermal equivalent over time. The measurement of this surface area makes it possible to evaluate the relaxing effects of the substances brought into contact beforehand with the dermal equivalent.

b) Protocol

Two series of attached dermal equivalents comprising normal human fibroblasts are prepared: a control series without any treatment and a series treated with the test compound (10 μM). The experiment is repeated three times.

The dermal equivalents are prepared as described in Asselineau et al., Exp. Cell. Res., 1985, 159, 536-539; Models in Dermatology, 1987, vol. 3, pp. 1-7, in the following proportions:

| | |
|---|---|
| MEM medium 1.76×, with or without compound | 45% |
| Foetal calf serum | 10% |
| NaOH (0.1N) | 5% |
| Acetic acid (1/1000) | 4% |
| Collagen | 26% |
| Fibroblasts | 10% |

The treated dermal equivalent differs from the control dermal equivalent in that 10 μM of the test compound are added thereto.

The collagen used is type I collagen (commercial solution). It is extracted from calf skin by acid hydrolysis and stored in an acidic medium at +4° C. It polymerizes naturally by reheating to 37° C. and by decreasing the degree of acidity. The collagen is dialysed beforehand against successive baths of water+acetic acid.

The protocol is as follows: the 1.76×MEM medium is introduced, in the presence of additives (1% glutamine, 1% non-essential amino acids, 1% sodium pyruvate, 1% fungizone and 1% penicillin/streptomycin), the foetal calf serum and the 0.1N NaOH solution, into a 50 ml centrifuge tube stored in crushed ice. The fibroblasts, isolated from human skin explants, are then added at the concentration of 1.5×10⁵ cells per 1 ml of culture medium.

A volume/volume mixture of collagen in acetic acid at 1/1000 is then slowly added, against the wall of the tube, so as to observe the appearance of a whitish cloud.

The combined product is then carefully mixed and distributed in the wells of a 12-well culture plate (Costar type, reference 3512) in a proportion of 2 ml of mixture per well. The final cell concentration is 3×10⁴ cells/dermal equivalent, with a final concentration of collagen of 1 mg/ml. The culture plate is then placed in an incubator at 37° C. with 5% CO₂.

Once formed after polymerization of the collagen, the dermal equivalents are left adhered to the culture support for 3 days and then detached from the support so that the contraction can begin. These detached dermal equivalents are taken out of the incubator in order to take images for the purpose of measuring their surface area, this being for each point of the kinetics of contraction (0, 4, 8 and 24 hours). They are immediately placed back in the incubator between each measurement point.

The evaluation of the spontaneous contraction of the treated (with the test compound) and control (without test compound) dermal equivalents is carried out by measuring their surface area at the different times mentioned after the beginning of the spontaneous contraction.

For this, a digital image is acquired for each treated or untreated dermal equivalent by means of a camera (CCD camera-Iris Sony DXC-107P) and the surface area is subsequently calculated on each image by means of an image analysis system (Zeiss Axiovision 3.0). There corresponds, to this surface area measurement, a percentage of contraction equal to the ratio of the surface areas according to the formula:
% contraction=(Sp−Si)/Sp×100
where: ‘Sp’ represents the surface area of a well of the culture plate; it corresponds to the total surface area of the dermal equivalent before contraction;
‘Si’ represents the surface area of the dermal equivalent at the time i of the kinetics of contraction.

c) Results:

Compounds C (Example 1), D (Example 4) and E (Example 5) and G (2′,3′-isopropylidene-5′-acetyladenosine; compound sold by Sigma under the reference I 4127) described above were tested according to the protocol described above and compared with adenosine and with isopropylideneadenosine.

The following results were obtained:

| Compound tested | % contraction |
|---|---|
| C | −30 |
| D | −21 |
| E | −26 |
| G | −30 |
| Adenosine | −15 |
| Isopropylideneadenosine | 0 |

Compounds C, D, E and G reduce the contraction of the fibroblasts by at least 21% on average over the duration of the experiment (tested at 10 μM), with respect to the control.

These compounds thus have a significant dermo-decontracting effect which is greater than that of adenosine (reduction of 15%). Isopropylideneadenosine has no dermo-decontracting effect.

Compounds C and G are particularly preferred due to their good result obtained.

### Example 7

### Skincare Composition

An oil-in-water emulsion is prepared which has the following composition:

| | |
|---|---|
| Glyceryl monostearate | 6.0% |
| Stearyl alcohol | 4.0% |
| Liquid petrolatum | 10.0% |
| Silicone oil | 5.0% |
| 5′-Acetyl-2′,3′-isopropylideneadenosine | 1.0% |
| (Compound G) | |
| Glycerol | 8.0% |
| Carboxyvinyl polymer, Carbopol type | 0.3% |
| Preservatives | 0.4% |
| Fragrance | 0.5% |
| Triethanolamine | 0.3% |
| Water | q.s. for 100% |

After application of the cream daily, the wrinkles of the face are softened.

Compound G can be replaced by Compound A or B.

### Example 8

### Skincare Composition

A gel for caring for the face is prepared which has the following composition:

| | |
|---|---|
| Compound C (Example 1) | 0.1% |
| Hydroxypropylcellulose (Klucel H, sold by | 1.00% |
| Hercules) | |
| Antioxidant | 0.05% |
| Isopropanol | 40.00% |
| Preservative | 0.30% |
| Water | q.s. for 100% |

This gel is obtained by mixing the constituents in water with the addition, lastly, of the gelling agent.

This gel can be applied twice daily; it is particularly suitable for application in the morning as it does not leave the skin greasy. A reduction in wrinkles is found after the application of the gel daily.

Compound C can be replaced by Compound F or G or D or E.

The above written description of the invention provides a manner and process of making and using it such that any person skilled in this art is enabled to make and use the same, this enablement being provided in particular for the subject matter of the appended claims, which make up a part of the original description and including:

(1) methods of using at least one adenosine derivative of formula (I): 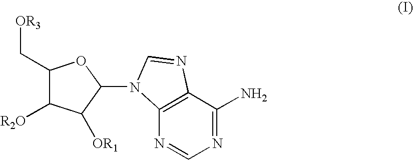
in which:
(a) R₁ and R₂, which are identical, denote a saturated linear C₁-C₆ or unsaturated linear C₂-C₆ or saturated or unsaturated branched C₃-C₆ hydrocarbon radical or form, together with the oxygen atoms to which they are attached, an isopropylidene radical;

and R₃ denotes:

(i) a —COR₄ group with R₄ denoting a saturated linear C₁-C₉ or unsaturated linear C₂-C₉ or saturated or unsaturated branched C₃-C₉ hydrocarbon radical optionally substituted by at least one group chosen from —OR′, —NR′R″, —COOR′, —CONR′R″, —CF₃, —F, —OCF₃, —CN or —NO₂;

or (ii) an ester group resulting from biotin;

or
(b) R₁ and R₂ form, together with the oxygen atoms to which they are attached, an isopropylidene radical; and R₃ denotes a saturated linear C₁-C₁₀ or unsaturated linear C₂-C₁₀ or saturated or unsaturated branched C₃-C₁₀ hydrocarbon radical optionally substituted by at least one group chosen from —OR′, —NR′R″, —COOR′, —CONR′R″, —CF₃, —F, —OCF₃, —CN or —NO₂;

R′ and R″ denoting a hydrogen atom or a saturated linear C₁-C₆ or unsaturated linear C₂-C₆ or saturated or unsaturated branched C₃-C₆ hydrocarbon radical optionally substituted by at least one group chosen from —OZ, —NZZ′ or —COOZ, Z and Z′ denoting, independently of one another, a hydrogen atom or a saturated linear C₁-C₆ or unsaturated linear C₂-C₆ or saturated or unsaturated branched C₃-C₆ hydrocarbon radical;

and its salts, optical isomers and solvates,

as agent for combating wrinkles, in particular expression wrinkles, and/or relaxing the skin and/or slackening the lines of the skin;

(2) Composition comprising, in a physiologically acceptable medium, at least one adenosine derivative of formula (II): 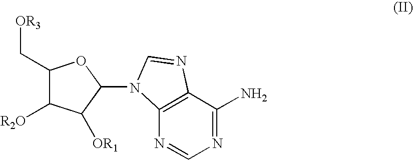
in which:
(a) R₁ and R₂, which are identical, denote a saturated linear C₁-C₆ or unsaturated linear C₂-C₆ or saturated or unsaturated branched C₃-C₆ hydrocarbon radical or form, together with the oxygen atoms to which they are attached, an isopropylidene radical;

and R₃ denotes:

(i) a —COR₄ group with R₄ denoting a saturated linear C₂-C₉ or unsaturated linear C₂-C₉ or saturated or unsaturated branched C₃-C₉ hydrocarbon radical optionally substituted by at least one group chosen from —OR′, —NR′R″, —COOR′, —CONR′R″, —CF₃, —F, —OCF₃, —CN or —NO₂;

or (ii) an ester group resulting from biotin;

or
(b) R₁ and R₂ form, together with the oxygen atoms to which they are attached, an isopropylidene radical; and R₃ denotes a saturated linear C₁-C₁₀ or unsaturated linear C₂-C₁₀ or saturated or unsaturated branched C₃-C₁₀ hydrocarbon radical optionally substituted by at least one group chosen from —OR′, —NR′R″, —COOR′, —CONR′R″, —CF₃, —F, —OCF₃, —CN or —NO₂;

R′ and R″ denoting a hydrogen atom or a saturated linear C₁-C₆ or unsaturated linear C₂-C₆ or saturated or unsaturated branched C₃-C₆ hydrocarbon radical optionally substituted by at least one group chosen from —OZ, —NZZ′ or —COOZ, Z and Z′ denoting, independently of one another, a hydrogen atom or a saturated linear C₁-C₆ or unsaturated linear C₂-C₆ or saturated or unsaturated branched C₃-C₆ hydrocarbon radical;

and its salts, optical isomers and solvates; and

(3) Compounds of formula (III): 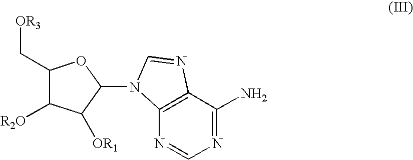
in which:
(a) R₁ and R₂, which are identical, denote a saturated linear C₁-C₆ or unsaturated linear C₂-C₆ or saturated or unsaturated branched C₃-C₆ hydrocarbon radical or else form, together with the oxygen atoms to which they are attached, an isopropylidene radical;
R₃ denotes:

(i) a —COR₄ group with R₄ denoting a linear C₃-C₉ hydrocarbon radical or a branched C₃-C₉ hydrocarbon radical which is saturated or unsaturated, optionally substituted by at least one group chosen from —OR′, —NR′R″, —CONR′R″, —CF₃, —F, —OCF₃, —CN or —NO₂, R₄ not denoting a 2-aminoethyl group;

or (ii) an ester group resulting from biotin;

or
(b) R₁ and R₂ form, together with the oxygen atoms to which they are attached, an isopropylidene radical; and R₃ denotes a linear C₂-C₁₀ hydrocarbon radical or a branched C₃-C₁₀ hydrocarbon radical which is saturated or unsaturated, optionally substituted by at least one group chosen from —NR′R″, —COOR′, —CONR′R″, —CF₃, —F, —OCF₃, —CN or —NO₂;

R′ and R″ denoting a hydrogen atom, a saturated linear C₁-C₆ or unsaturated linear C₂-C₆ hydrocarbon radical or a saturated or unsaturated branched C₃-C₆ radical optionally substituted by at least one group chosen from —OZ, —NZZ′ or —COOZ, Z and Z′ denoting, independently of one another, a hydrogen atom or a saturated linear C₁-C₆ or unsaturated linear C₂-C₆ or saturated or unsaturated branched C₃-C₆ hydrocarbon radical;

and their salts, optical isomers and solvates.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A method for combating wrinkles, relaxing skin and/or slackening the lines of the skin comprising applying to skin at least one adenosine derivative of formula (I): 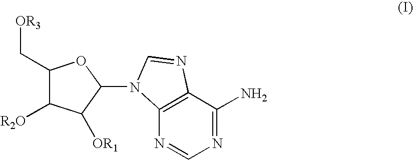
in which:
(a) R₁ and R₂, which are identical, denote a saturated linear C₁-C₆ or unsaturated linear C₂-C₆ or saturated or unsaturated branched C₃-C₆ hydrocarbon radical or form, together with the oxygen atoms to which they are attached, an isopropylidene radical; and R₃ denotes:
(i) a —COR₄ group with R₄ denoting a saturated linear C₁-C₉ or unsaturated linear C₂-C₉ or saturated or unsaturated branched C₃-C₉ hydrocarbon radical optionally substituted by at least one group chosen from —OR′, —NR′R″, —COOR′, —CONR′R″, —CF₃, —F, —OCF₃, —CN or —NO₂; or
(ii) an ester group resulting from biotin; or
(b) R₁ and R₂ form, together with the oxygen atoms to which they are attached, an isopropylidene radical; and R₃ denotes a saturated linear C₁-C₁₀ or unsaturated linear C₂-C₁₀ or saturated or unsaturated branched C₃-C₁₀ hydrocarbon radical optionally substituted by at least one group chosen from —OR′, —NR′R″, —COOR′, —CONR′R″, —CF₃, —F, —OCF₃, —CN or —NO₂; and
R′ and R″ denoting a hydrogen atom or a saturated linear C₁-C₆ or unsaturated linear C₂-C₆ or saturated or unsaturated branched C₃-C₆ hydrocarbon radical optionally substituted by at least one group chosen from —OZ, —NZZ′ or —COOZ, Z and Z′ denoting, independently of one another, a hydrogen atom or a saturated linear C₁-C₆ or unsaturated linear C₂-C₆ or saturated or unsaturated branched C₃-C₆ hydrocarbon radical;
and its salts, optical isomers and solvates,
in an amount sufficient to combat wrinkles, relax skin and/or slacken the lines of the skin.

2. The method according to claim 1, wherein in the adenosine derivative:
R₁ and R₂ form, together with the oxygen atoms to which they are attached, an isopropylidene radical; and
R₃ denotes:
(i) a saturated linear C₁-C₁₀ or unsaturated linear C₂-C₁₀ or saturated or unsaturated branched C₃-C₁₀ hydrocarbon radical;
(ii) a —COR₄ group with R₄ denoting a linear C₁-C₉ hydrocarbon radical or a branched C₃-C₉ hydrocarbon radical which is saturated or unsaturated; or
(iii) an ester group resulting from biotin.

3. The method according to claim 1, wherein in the adenosine derivative:
R₁ and R₂ form, together with the oxygen atoms to which they are attached, an isopropylidene radical; and
R₃ denotes:
(i) a linear C₁-C₁₀ hydrocarbon radical;
(ii) a —COR₄ group with R₄ denoting a saturated linear C₁-C₉ hydrocarbon radical; or
(iii) an ester group resulting from biotin.

4. The method according to claim 1, wherein in the adenosine derivative:
R₁ and R₂ form, together with the oxygen atoms to which they are attached, an isopropylidene radical;
R₃ denotes:
a —COR₄ group with R₄ denoting a linear C₁-C₉ hydrocarbon radical; or
an ester group resulting from biotin.

5. The method according to claim 1, wherein the adenosine derivative is selected from the group consisting of:
2′,3′-isopropylidene-5′-butanoyladenosine;
2′,3′-isopropylidene-5′-octanoyladenosine;
2′,3′-isopropylidene-5′-biotinoyladenosine;
2′,3′-isopropylidene-5′-ethyladenosine;
2′,3′-isopropylidene-5′-octyladenosine;
2′,3′-dimethyl-5′-butanoyladenosine; and
2′,3′-isopropylidene-5′-acetyladenosine.

6. The method according to claim 1, wherein the skin to which the adenosine derivative is applied is skin of the face and/or of the forehead.

7. The method according to claim 1, wherein the adenosine derivative is in a composition comprising a physiologically acceptable medium.

8. A composition comprising, in a physiologically acceptable medium, at least one adenosine derivative of formula (II): 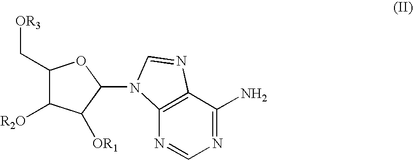
in which:
(a) R₁ and R₂, which are identical, denote a saturated linear C₁-C₆ or unsaturated linear C₂-C₆ or saturated or unsaturated branched C₃-C₆ hydrocarbon radical or form, together with the oxygen atoms to which they are attached, an isopropylidene radical;
and R₃ denotes:
(i) a —COR₄ group with R₄ denoting a saturated linear C₂-C₉ or unsaturated linear C₂-C₉ or saturated or unsaturated branched C₃-C₉ hydrocarbon radical optionally substituted by at least one group chosen from —OR′, —NR′R″, —COOR′, —CONR′R″, —CF₃, —F, —OCF₃, —CN or —NO₂; or
(ii) an ester group resulting from biotin;
or
(b) R₁ and R₂ form, together with the oxygen atoms to which they are attached, an isopropylidene radical; and R₃ denotes a saturated linear C₁-C₁₀ or unsaturated linear C₂-C₁₀ or saturated or unsaturated branched C₃-C₁₀ hydrocarbon radical optionally substituted by at least one group chosen from —OR′, —NR′R″, —COOR′, —CONR′R″, —CF₃, —F, —OCF₃, —CN or —NO₂; and
R′ and R″ denoting a hydrogen atom or a saturated linear C₁-C₆ or unsaturated linear C₂-C₆ or saturated or unsaturated branched C₃ -C₆ hydrocarbon radical optionally substituted by at least one group chosen from —OZ, —NZZ′ or —COOZ, Z and Z′ denoting, independently of one another, a hydrogen atom or a saturated linear C₁-C₆ or unsaturated linear C₂-C₆ or saturated or unsaturated branched C₃-C₆ hydrocarbon radical;
and its salts, optical isomers and solvates.

9. The composition according to claim 8, wherein in the adenosine derivative:
R₁ and R₂ form, together with the oxygen atoms to which they are attached, an isopropylidene radical; and R₃ denotes:
(i) a saturated linear C₁-C₁₀ or unsaturated linear C₂-C₁₀ or saturated or unsaturated branched C₃-C₁₀ hydrocarbon radical;
(ii) a —COR₄ group with R₄ denoting a linear C₂-C₉ hydrocarbon radical or a branched C₃-C₉ hydrocarbon radical which is saturated or unsaturated; or
(iii) an ester group resulting from biotin.

10. The composition according to claim 8, wherein in the adenosine derivative:
R₁ and R₂ form, together with the oxygen atoms to which they are attached, an isopropylidene radical; and R₃ denotes:
(i) a linear C₁-C₁₀ hydrocarbon radical;
(ii) a —COR₄ group with R₄ denoting a saturated linear C₂-C₉ hydrocarbon radical; or
(iii) an ester group resulting from biotin.

11. The composition according to claim 8, wherein in the adenosine derivative:
R₁ and R₂ form, together with the oxygen atoms to which they are attached, an isopropylidene radical; and
R₃ denotes:
a —COR₄ group with R₄ denoting a linear C₂-C₉ hydrocarbon radical;
or an ester group resulting from biotin.

12. The composition according to claim 8, wherein the adenosine derivative is selected from the group consisting of:
2′,3′-isopropylidene-5′-butanoyladenosine;
2′,3′-isopropylidene-5′-octanoyladenosine;
2′,3′-isopropylidene-5′-biotinoyladenosine;
2′,3′-isopropylidene-5′-ethyladenosine;
2′,3′-isopropylidene-5′-octyladenosine; and
2′,3′-dimethyl-5′-butanoyladenosine.

13. The composition according to claim 8, wherein the composition is a cosmetic or dermatological composition.

14. The composition according to claim 8, wherein the composition further comprises at least one ingredient selected from the group consisting of oils, waxes, emulsifiers, gelling agents, film-forming polymers, preservatives, fragrances, fillers, UV screening agents, bactericides, odour absorbers, dyestuffs, hydrophilic or lipophilic active principles, plant extracts, antioxidants, and mixtures thereof.

15. Compounds of formula (III): 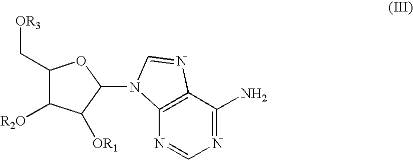
in which:
(a) R₁ and R₂, which are identical, denote a saturated linear C₁-C₆ or unsaturated linear C₂-C₆ or saturated or unsaturated branched C₃-C₆ hydrocarbon radical or else form, together with the oxygen atoms to which they are attached, an isopropylidene radical; and
R₃ denotes:
(i) a —COR₄ group with R₄ denoting a linear C₃-C₉ hydrocarbon radical or a branched C₃-C₉ hydrocarbon radical which is saturated or unsaturated, optionally substituted by at least one group chosen from —OR′, —NR′R″, —CONR′R″, —CF₃, —F, —OCF₃, —CN or —NO₂, R₄ not denoting a 2-aminoethyl group; or
(ii) an ester group resulting from biotin;
or
(b) R₁ and R₂ form, together with the oxygen atoms to which they are attached, an isopropylidene radical;
R₃ denotes a linear C₂-C₁₀ hydrocarbon radical or a branched C₃-C₁₀ hydrocarbon radical which is saturated or unsaturated, optionally substituted by at least one group chosen from —NR′R″, —COOR′, —CONR′R″, —CF₃, —F, —OCF₃, —CN or —NO₂; and
R′ and R″ denoting a hydrogen atom, a saturated linear C₁-C₆ or unsaturated linear C₂-C₆ hydrocarbon radical or a saturated or unsaturated branched C₃-C₆ radical optionally substituted by at least one group chosen from —OZ, —NZZ′ or —COOZ, Z and Z′ denoting, independently of one another, a hydrogen atom or a saturated linear C₁-C₆ or unsaturated linear C₂-C₆ or saturated or unsaturated branched C₃-C₆ hydrocarbon radical;
and their salts, optical isomers and solvates.

16. The compound according to claim 15, wherein in the adenosine derivative:
R₁ and R₂ form, together with the oxygen atoms to which they are attached, an isopropylidene radical; and
R₃ denotes:
(i) a linear C₂-C₁₀ hydrocarbon radical or a branched C₃-C₁₀ hydrocarbon radical which is saturated or unsaturated;
(ii) a —COR₄ group with R₄ denoting a linear C₃-C₉ hydrocarbon radical or a branched C₃-C₉ hydrocarbon radical which is saturated or unsaturated; or
(iii) an ester group resulting from biotin.

17. The compound according to claim 15, wherein in the adenosine derivative:
R₁ and R₂ form, together with the oxygen atoms to which they are attached, an isopropylidene radical; and
R₃ denotes:
(i) a linear C₂-C₁₀ hydrocarbon radical;
(ii) a —COR₄ group with R₄ denoting a saturated linear C₃-C₉ hydrocarbon radical; or
(iii) an ester group resulting from biotin.

18. The compound according to claim 15, wherein in the adenosine derivative:
R₁ and R₂ form, together with the oxygen atoms to which they are attached, an isopropylidene radical; and
R₃ denotes:
a —COR₄ group with R₄ denoting a linear C₃-C₉ hydrocarbon radical; or
an ester group resulting from biotin.

19. The compound according to claim 15, wherein the adenosine derivative is selected from the group consisting of:
2′,3′-isopropylidene-5′-butanoyladenosine;
2′,3′-isopropylidene-5′-octanoyladenosine;
2′,3′-isopropylidene-5′-biotinoyladenosine;
2′,3′-isopropylidene-5′-ethyladenosine;
2′,3′-isopropylidene-5′-octyladenosine; and
2′,3′-dimethyl-5′-butanoyladenosine.
